(19) United States
(12) Patent Application Publication
(10) Pub. No.: US 2013/0225900 A1
(43) Pub. Date: Aug. 29, 2013

(54) Method and Device for Closure of Intraluminal Perforations

(71) Applicants: Anthony N. Kalloo, Baltimore, MD (US); Mouen A. Khashab, Towson, MD (US)

(72) Inventors: Anthony N. Kalloo, Baltimore, MD (US); Mouen A. Khashab, Towson, MD (US)

(21) Appl. No.: 13/877,387

(22) PCT Filed: Oct. 3, 2011
(86) PCT No.: PCT/US11/54641

§ 371 Date: May 7, 2013

Related U.S. Application Data
(60) Provisional application No. 61/389,326, filed on Oct. 4, 2010.
(60) Provisional application No. 61/522,529, filed on Aug. 11, 2011.

(51) Int. Cl. A61B 17/00; A61B 18/04; A61N 5/02; A61N 5/10; A61N 5/06

(52) U.S. Cl. CPC A61B 17/0057; A61N 5/10; A61N 5/06; A61N 5/02; A61B 18/04
USPC 600/1; 606/153; 606/154; 606/139; 606/2; 606/27; 606/33; 604/522; 604/506

(57) ABSTRACT

A closure device having evertable arms for capturing and everting tissue and method for its use with an invasive scope, such as an endoscope, for full-thickness closure of perforations and leaks in the walls of an intraluminal bodily space.

### BACKGROUND OF THE INVENTION

1. Field of the Invention

The invention relates to surgical devices and methods for closing leaks and perforations in and through the walls of bodily lumens.

2. Background Information

Iatrogenic perforation is a major complication, which is difficult to endoscopically manage, and, if large, inevitably requires surgical repair. Perforations or leaks in the walls of internal organs and vessels may occur naturally (e.g., through rupture of herniated tissue) or formed unintentionally (e.g., as a result of a surgical procedure, such as tissue resection). In the latter respect, increasing practice of endoscopic therapies can increase the incidence of iatrogenic perforation in luminal tissues such as the gastrointestinal (GI) tract. When a perforation is formed in the stomach or intestines, spillage of the stomach contents, intestinal contents or other bodily fluids into the adjacent body cavity can occur, providing a potentially deadly environment for infection.

To minimize leakage of bowel contents, risk for severe peritonitis, and abscess formation, prompt repair of the perforation site is highly desirable. An urgent surgical closure remains the primary standard treatment for acute perforation. Devices for use in closing perforations and leaks generally involve deployment of clips, sutures, adhesives, patches or tissue anchors to join adjacent tissues together around the opening. The closure device is often introduced to the surgical site endoscopically or laparoscopically, and used in conjunction with tissue graspers or other devices to orient the tissue for closure.

However, the efficacy of such devices has been suboptimal. For example, endoscopic repair with metallic mucosal clips (see, e.g., K. F. Binmoeller, et al., Gastrointest Enclosc 39:172-174 (1993), A. Charabaty-Pishvaian and F. Al-Kawas, South Med J 97:190-193 (2004), and S. S. Dhalla, Endoscopic repair of a colonic perforation following polypectomy using an endoclip, Can J Gastroenterol 18:105-106 (2004)) and an omental patch has been described as a convenient therapeutic option. However, application of mucosal clips with single-layer tissue closure (e.g., muscle layer to muscle layer) is of limited usefulness and best suited for repairing small, linearly shaped defects. An omental patch is convenient for repair of lesions involving the anterior GI wall, but is difficult to apply to other GI organs and to more distal sites. As such, use of the omental patch has been limited to the stomach (see, e.g., Hashiba, A. M., et al., Gastrointest Endosc 54:500-504 (2001)).

A need therefore remains for an endoscopic closure device which can be used on a full thickness basis (through the organ wall, mucosa, submucosa, muscular propria layer and serosa) for treatment of intraluminal leaks and perforations, including gastrointestinal perforations, fistulas and anastomotic leaks, as well as perforations in other luminal tissues, including esophageal, gastric, intestinal and colonic perforations.

### SUMMARY OF THE INVENTION

The invention provides a device and method for its endoscopic use for full-thickness closure of leaks and perforations in the wall of an intraluminal bodily space, such as the gastrointestinal tract.

The device has a plurality of elongated arms defining a conical or umbrella-like shell, and a central axis including a central apparatus attachable to a pusher or guidewire. At least two of the elongated arms terminate distally in a tissue-capturing structure, such as barbs or hooks.

According to one aspect of the invention, the arms are formed of a biocompatible flexible resilient material, preferably a shape-memory material. The material allows the shell to assume a compressed configuration with the arms folded down along the central axis and an expanded configuration with the arms extending away from the central axis. Placing the device into the first configuration allows the shell to be compressed into a through-the-scope tool; e.g., a delivery catheter. Delivery of the shell out of the proximal end of the tool allows the shell to assume an expanded configuration as the arms move outwardly.

According to a further aspect of the invention, a foldable biocompatible material (e.g., a mesh) may be disposed between the support arms to enhance their stability when in their expanded configuration, similar to a cover over the arms of an umbrella.

According to a further aspect of the invention, the tissue capture elements are treated to promote wound healing. It will be appreciated that the closure device is intended for surgical use only, and is neither intended nor adapted to be implantable. However, contact between the tissue capture elements and the tissues of the endoluminal space creates an environment where delivery or elution of wound healing promoting molecules could be useful.

According to a further aspect of the invention, the central axis of the device is releasably attachable to a pusher structure, such as a guidewire.

According to a further aspect of the invention, the device includes a system for its introduction into a luminal space to the site of a perforation or leak to be treated. Such system preferably includes an endoscope or comparable instrument for insertion into an intraluminal space (e.g., a laparoscope) and a loader catheter, into which the device may be provided pre-loaded.

For use according to the invention, the compressed shell of the device is inserted into a delivery tool, such as a loader catheter. The loader catheter can be introduced through the accessory channel of a therapeutic endo scope and advanced through the perforation under endoscopic visualization. The shell is advanced out of the loader catheter and through the perforation. Retraction of the shell causes the tissue-capturing structure to engage tissue surrounding the perforation or leak. As the shell is pulled back through the perforation, the shell everts with the tissue, pulling the full thickness of the latter back with it.

According to one aspect of the invention, the fully retracted tissue is closed with a mechanical device, such as a clip or suture. Alternatively, the tissue may be closed by sealing through application of energy or an adhesive thereto.

According to a further aspect of the invention, the entire everted and retracted shell is withdrawn from the body following treatment. Alternatively, the tissue capture elements are detached from the shell (e.g., by snipping them off the arms) and remain in the body.

### BRIEF DESCRIPTION OF THE DRAWINGS

FIG. 1A depicts a closure device of the invention in its compressed configuration.

FIG. 1B depicts a cross-sectional view of a closure device of the invention.

FIG. 2 depicts a closure device of the invention being deployed from the proximal end of a loader catheter provided through an endoscope.

FIG. 3 depicts a closure device of the invention in its expanded configuration, as engaged with the wall of a bodily lumen.

### DETAILED DESCRIPTION OF THE INVENTION

### Materials for Use in the Devices of the Invention.

The term endoscope, as used herein, should be construed as including all types of invasive instruments, flexible or rigid, having scope features. These include, but are not limited to, colonoscopes, gastroscopes, laparoscopes, and rectoscopes. Similarly, the use of “endoscopic” is to be construed as referring to all types of invasive surgical scopes.

An endoscopic system, and a shell device according to this invention will be of particular use in full thickness closure of a perforation or leak, such as arising in for example, but without intending to be limiting, the bowel, rectum, appendix, gallbladder, uterus, stomach, esophagus, and other luminal cavities of the body.

An endoscope with accessory channels such as the CF2T-160L from Olympus, or DUETTE® from Cook Medical may be used to deliver a closure device of the invention.

Preferred materials for use in the supporting arms of the closure device of the invention are shape memory materials, such as NITINOL®, from Medtronic. NITINOL® is a “shape memory alloy” based on nickel and titanium with moderate deformability. Other shape memory materials having greater deformability may also be utilized, so long as (1) the arms of the closure device may be moved from an expanded to an everted configuration as described hereinbelow; and (2) the hooks or other capture elements on the proximal end of one or more of the supporting arms are sufficiently rigid to engage and remain secured in the serosa/adventitial wall of a luminal space when subjected to pulling force according to the invention.

In clinically suitable applications, the tissue capture elements may be detached from the shell and left behind in the intraluminal space. For such applications, the material used at least in the tissue capture elements, if not also the entire shell, may be a polymer with bioresorbable, thermoplastically deformable shape memory characteristics, such as those described in U.S. Patent Publication No. 2010/0262182, or a biological material, such as collagen. In the latter embodiments, the capture elements (e.g., hooks) at the proximal ends of the supporting arms may be formed of the same material as the supporting arms, or a different material. They may also be treated to with molecules known in the art to promote epithelialisation and wound healing (e.g., chitosan, steroids and the like).

If present, the shell cover may be of any biocompatible material including, for leave behind applications, a bioabsorbable material (shape memory or not), such as polylactic acid (PLA), poly-glycolic acid (PGA), polylactide-co-glycolide, isomers and combinations thereof. The shell cover may also be formed of a biological material (e.g., collagen).

### Structure of the Devices of the Inventions.

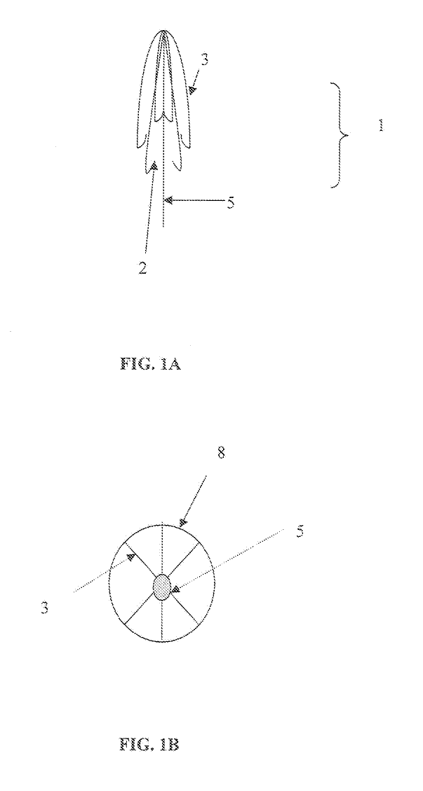

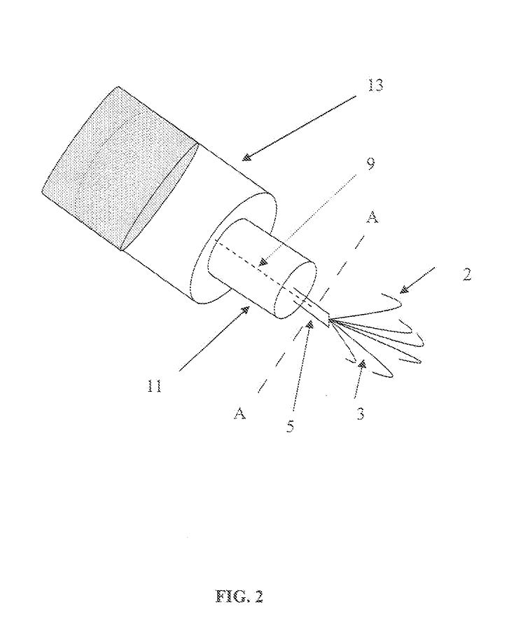

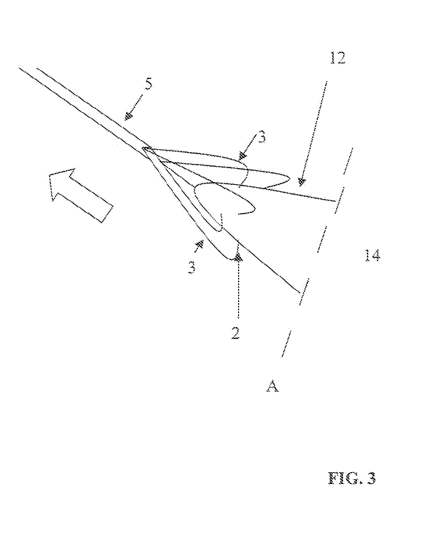

Turning to FIG. 1, a closure device 1 of the invention is shown in its compressed configuration. Arms 3 are attached at their proximal end to a central apparatus 5, which consists of a rod, and have hooks 2 at their distal ends. Arms 3 are moveable (substantially evertable) from a compressed configuration to an expanded, everted one (compare FIGS. 2 and 3); e.g., by bending or rotating near their attachment point to central apparatus 5 or along a mechanical pivot point.

As shown in FIG. 2, central apparatus 5 is disposed through a loader catheter 11 and is attached at its proximal end (preferably releasably) or through a bore (not shown) disposed axially through central apparatus 5 to a pusher structure 9. As shown in FIGS. 1 and 2, six arms are present; however, it will be apparent to those of ordinary skill in the art that a lesser or greater number of arms may be utilized; e.g., from at least 2 up to 20 arms, preferably 4-12 arms, most preferably 4-8 arms. FIG. 1B also depicts the optional shell cover 8 disposed over arms 3.

Pusher structure 9 is shown in phantom in FIG. 2 as a guidewire, but may be any equivalent structure, such as a rod or catheter. If a guidewire, pusher 9 may be a conventional wire or a self-centering guidewire, to facilitate insertion of the closure device through a perforation. Where a self-centering guidewire is utilized, the shell defined by arms 3 of closure device 1 need not be as large in diameter as compared to the diameter of the perforation being treated. In either embodiment, 5 central apparatus and pusher 9 are insertable through a loader catheter 11 for an endoscope 13.

As noted, in surgical use, the closure device is inserted into loader catheter 11 in its compressed configuration. Tension exerted by contact between the inner diameter of loader catheter 11 against arms 3 retains them in a downward attitude, compressed toward the central axis of device 1. Endoscope 13 of is advanced to the site of the perforation to be treated in a body cavity and loader catheter 11 advanced through the perforation across line A-A, as shown in FIG. 2. The closure device is advanced distally out of loader catheter 11, by operation of pusher apparatus 9. As the shell is advanced distally out of loader catheter 11, arms 3 deploy into the expanded configuration as the tension on arms 3 is released. As such, the arms 3 do not become fully deployed from the proximal end of the loader catheter until they have passed completely out of the loader catheter and through the perforation. Loader catheter 11 is then retracted.

Once arms 3 are fully deployed in an expanded configuration, device 1 is pulled back toward the perforation (in the direction indicated by the block arrow of FIG. 3) until the hooks are captured into the serosal/adventitial surface of the organ wall 12 around the perforation site. Arms 3 then become everted out of the intraluminal space 14 through the perforation along line A-A by retraction of the central apparatus toward and back through the perforation. As device 1 is pulled backward, arms 3 and hooks 2 exert a pulling force on the serosal/adventitial surface 12, everting it into the luminal space.

Eversion of the tissue surrounding the perforation closes it and provides a treatment surface for sealing of the perforation. Sealing can be accomplished by application of a clip, suture or staple to the everted tissue. Alternatively, sealing may be accomplished for relatively small perforations or leak by application of energy to the everted tissue (causing tightening of tissue collagen; e.g., by operation of one or more radiofrequency, RF, electrodes disposed on arms 3 or introduced to the treatment site through endoscope 13) or by application of a biocompatible surgical adhesive. Radiation applied to the tissue may include, by way of illustration, visible light, infrared radiation, microwave radiation, radio waves, very low frequency (VLF) radiation, extremely low frequency (ELF) radiation, and thermal radiation.

If the perforation is sufficiently small or symmetrical, eversion alone may be sufficient to close it. According to one embodiment, when fully deployed, arms 3 define a shell having a diameter of less than twice the size of the defect to be closed. For larger lesions, full-thickness closure of the everted wall tissue is preferably performed using a suturing device and/or endoscopic clips. Once treatment is complete, hooks 2 may be pulled out of the tissue or detached (e.g., by clipping) to allow removal of the device from the luminal space.

The following examples are provided to further illustrate the advantages and features of the present invention, but are not intended to limit the scope of the invention. While they are typical of those that might be used, other procedures, methodologies, or techniques known to those skilled in the art may alternatively be used.

### EXAMPLE I

### Endoscopic Sealing of a Gi Perforation

For surgical preparation, the mucosa of the upper gastric body is lavaged with sterilized water and povidone-iodine via an accessory channel of the endoscope.

A pig with perforations or leaks created by incisions through the anterior wall of the GI tract is a suitable model for demonstration of the invention. After the incision, the endoscope is inserted into the peritoneal cavity to confirm a full-thickness incision of the gastric wall of at least 1 cm in diameter. To close each incision, treatment is performed with a closure device of the invention having a 1.5 cm diameter shell according to the above-described method.

Oral intake is withheld during the immediate postanesthesia recovery period (about 6 hours). The pigs may be fed a softened diet for several days and then a normal diet as tolerated. The pigs are preferably also treated with antibiotics for 5 days. One week after the procedure, follow-up endoscopy is performed, and the pig euthanized. A necropsy maybe performed to assess the perforation sites both intraluminally and intraperitoneally.

Although the invention has been described with reference to the above example, it will be understood that modifications and variations are encompassed within the spirit and scope of the invention. Accordingly, the invention is limited only by the following claims.

1. A device for full-thickness closure of intraluminal perforations, the device having a central axis and further comprising:
(a) a plurality of substantially evertable elongated arms defining a conical shell, wherein at least two of the arms terminate distally in a tissue-capturing structure; and,
(b) a central apparatus disposed along the central axis secured to at least one of the elongated arms.

2. The device of claim 1, wherein the device comprises between about 2 to 20 elongated arms.

3. The device of claim 2, wherein the device comprises between about 4 to 8 elongated arms.

4. The device of claim 1, wherein between about 2 to 8 of the arms terminate distally in the tissue-capturing structure.

5. The device of claim 1, wherein the distally terminating arms are configures as barbs or hooks.

6. The device of claim 1, wherein the plurality of elongated arms are formed of a deformably resilient shape memory material.

7. The device of claim 6, wherein the deformably resilient shape memory material is an alloy, polymer or combination thereof.

8. The device of claim 4, wherein the tissue-capturing structure is formed of a bioresorbable material.

9. The device of claim 4, wherein the tissue-capturing structure is treated to promote wound healing.

10. The device of claim 6, wherein the arms terminating distally in the tissue-capturing structure are formed of a deformably resilient shape memory alloy.

11. The device of claim 1, wherein the arms are evertable along a pivot point.

12. The device of claim 1, wherein the device further comprises a cover disposed over the conical shell.

13. The device of claim 11, wherein the cover is a mesh or lattice.

14. The device of claim 1, wherein the plurality of elongated arms define a conical shell having a diameter of less than twice that of a perforation within which the device is fully deployed.

15. The device of claim 1, wherein the plurality of elongated arms are operably coupled to a pusher structure to facilitate deployment of the device.

16. The device of claim 15, wherein the pusher structure is configured as a guidewire, rod or catheter.

17. The device of claim 16, wherein the pusher structure is a guidewire.

18. The device of claim 17, wherein the guidewire is self-centering.

19. The device of claim 18, wherein the shell has a diameter less than that of a perforation within which the device is fully deployed.

20. The device of claim 15, wherein the pusher structure and the central apparatus are adapted to be insertable through a lumen of a catheter or an endoscope.

21. A system for full-thickness closure of intraluminal perforations, the system comprising:
(a) a closure device according to claim 1; and,
(b) a loader catheter,
wherein the arms of the closure device are compressed toward the central axis for insertion into the loader catheter, and evertably deploy into an expanded configuration when advanced distally out of the loader catheter.

22-40. (canceled)

41. A method for full-thickness closure of intraluminal perforations, the method comprising:
(a) advancing a system according to claim 21 into the perforation through an invasive surgical scope;
(b) deploying the device out of the loader catheter;
(c) retracting the device toward the perforation until the plurality of elongated aims are captured into the serosal/adventitial surface of tissue around the perforation site;
(d) continuing to retract the device to evert the arms until the tissue becomes everted into the luminal space by pulling force exerted through the everted aims; and,
(e) sealing the perforation.

42. The method of claim 41, further comprising retracting the device into the catheter.

43. The method of claim 42, further comprising detaching the at least two elongated arms terminating distally in the tissue-capturing structure.

44. The method of claim 42, wherein the device further comprises a cover disposed over the conical shell.

45. The method of claim 44, wherein the tissue-capturing structure is treated to promote wound healing.

46. The method of claim 45, wherein the tissue-capturing structure is detached and remains in the tissue surrounding the perforation after removal of the device.

47. The method of claim 41, wherein (e) comprises application of a closure device.

48. The method of claim 41, wherein the closure device is a tissue clip, a suture or a staple.

49. The method of claim 41, wherein (e) comprises application of radiation.

50. The method of claim 49, wherein the radiation is ionizing or non-ionizing radiation.

51. The method of claim 50, wherein the radiation is selected from visible light, infrared radiation, microwave radiation, radio waves, very low frequency (VLF) radiation, extremely low frequency (ELF) radiation, and thermal radiation.

52. The method of claim 41, wherein (e) comprises application of a bioadhesive.

53. The method of claim 41, wherein (e) comprises application of a pharmaceutical agent.

54. The method of claim 53, wherein the agent is a biomolecules, protein, oligonucleotide, hormone, steroid, growth factor, transcription factor, cell, matrix protein, chemical compound, or combination thereof.

55. The method of claim 41, wherein the shell has a diameter of less than twice that of the perforation within which the device is fully deployed.

56. The method of claim 41, wherein the shell has a diameter less than that of the perforation within which the device is fully deployed.
